(19) United States
(12) Patent Application Publication
(10) Pub. No.: US 2010/0152637 A1
(43) Pub. Date: Jun. 17, 2010

(54) ORTHOPEDIC SHOE HAVING HEIGHT-ADJUSTABLE CALF GUARD

(75) Inventor: Chih-Chuan Wang, Taichung County (TW)

Correspondence Address: BROWDY AND NEIMARK, P.L.L.C.;624 NINTH STREET, NW, SUITE 300, WASHINGTON, DC, 20001-5303, US

(73) Assignee: KAO CHEN ENTERPRISE CO., LTD., Taichung Hsien (TW)

(21) Appl. No.: 12/413,728

(22) Filed: Mar. 30, 2009

(30) Foreign Application Priority Data
Dec. 17, 2008 (TW) 97149296

(51) Int. Cl. A61F 5/01

(52) U.S. Cl. 602/23

(57) ABSTRACT

An orthopedic shoe includes a shoe body provided with shoe straps for fastening to a user's foot, a track vertically affixed to the shoe body, a calf guard vertically slidably coupled to the track, and a locking lever pivoted to the calf guard and biasable between a locking position to lock the calf guard to the track and an unlocking position to unlock the calf guard from the track for allowing adjustment of the elevation of the calf guard relative to the track.

### BACKGROUND OF THE INVENTION

1. Field of the Invention

The present invention relates generally to orthopedic shoes and more particularly, to an orthopedic shoe that allows adjustment of the height of the calf guards.

2. Description of the Related Art

Due to busy traffic and complicated road conditions, people's legs injuries, such as bone breaking, ankle sprain, contusion, etc., are frequently seen. Leg wound can be healed after proper treatment. In case of a bone breaking, it takes a long time to resume normal mobility after through a rehabilitation process. To protect the injured leg and to give it a support, it is necessary to wear an orthopedic shoe. Taiwan Patent No. 480997 discloses an orthopedic shoe, which comprises a plastic guard shell and a rubber sole located on the bottom side of the plastic guard shell.

The aforesaid prior art orthopedic shoe has a fixed size, not adjustable to fit different users. If the orthopedic shoe does not fit the user's leg perfectly, the orthopedic shoe may slip when the user is walking.

Therefore, it is desirable to provide an orthopedic shoe that can be conveniently adjusted to fit the user's leg.

### SUMMARY OF THE INVENTION

The present invention has been accomplished under the circumstances in view. It is one objective of the present invention to provide an orthopedic shoe having a height-adjustable calf guard, which is conveniently adjustable subject to the condition of the user's leg for perfect fit.

To achieve this objective of the present invention, an orthopedic shoe comprises a shoe body, at least one strap inserted through the shoe body for fastening the shoe body to a user's foot, and at least one support device mounted on the shoe body to support the user's leg. Each support device comprises a track affixed to the shoe body, a calf guard coupled to the track and movable relative to the track and having a receiving chamber adapted for receiving the track, and a locking lever pivoted to the calf guard and biasable relative to the calf guard between a locking position and an unlocking position. The locking lever comprises a cam, which can be forced into engagement with the track to lock the calf guard to the track when the locking lever is biased from the unlocking position to the locking position, or disengaged from the track for allowing movement of the calf guard relative to the track when the locking lever is biased from the locking position to the unlocking position.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a perspective view of an orthopedic shoe in accordance with a preferred embodiment of the present invention;

FIG. 2 is a sectional view of a part of the orthopedic shoe of the present invention, showing the locking lever in a first (locking) position;

FIG. 3 is similar to FIG. 2, but showing the locking lever biased to a second (unlocking) position, and

FIG. 4 is similar to FIG. 2 but showing that the elevation of the calf guard is adjusted lower.

### DETAILED DESCRIPTION OF THE INVENTION

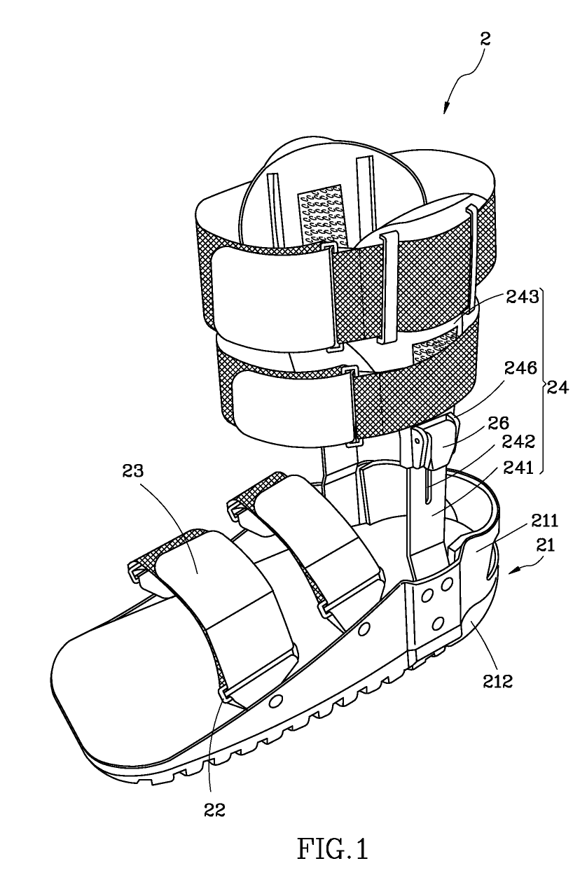

Referring to FIG. 1, an orthopedic shoe 2 in accordance with a preferred embodiment of the present invention is shown comprising a shoe body 21 having an outside counter 211. The shoe body 21 has its top side covered with a shock-absorbing layer of foam material to protect the user's leg from vibration injury. The shoe body 21 has its bottom side mounted with a sole 212. The sole 212 is made of rubber. The sole 212 has an anti-slip structure on its one side, namely, the bottom side opposite to the top side that is bonded to the shoe body 21. By means of the anti-slip structure, the orthopedic shoe 2 does not slip when the user is walking.

Strap frames 22 are bilaterally symmetrically provided at the front part of the outside counter 211. Straps 23 are inserted through the strap frames 22 for fastening the user's leg to the shoe body 21.

Two support devices 24 are bilaterally affixed to the rear part of the outside counter 211. Each support device 24 comprises a track 241 and a calf guard 243, a guide pin 25, and a locking lever 26. The track 241 has one end fixedly fastened to the outside counter 211. The other end of the track 241 is a free end. The track 241 can be affixed to the outside counter 211 by screws, rivets, or an adhesive. According to the present preferred embodiment, the track 241 is riveted to the outside counter 211. Further, the track 241 has a sliding slot 242 that cuts through the track 241 and extends along the length of the track 241. The calf guard 243 is adapted to protect and hold the user's leg. The calf guard 243 has a receiving chamber 244, an opening 245 disposed in communication with the receiving chamber 244, and a plurality of, for example, two protruding portions 246 disposed at two opposite lateral sides relative to the opening 245. By means of the receiving chamber 244, the calf guard 243 is sleeved onto the track 241. The guide pin 25 is riveted to the calf guard 243 and inserted through the sliding slot 242 to guide sliding movement of the calf guard 243 along the sliding slot 242 and to prohibit disconnection of the calf guard 243 from the track 241. The locking lever 26 is pivotally connected to the protruding portions 246 of the calf guard 243, having a cam 261. The locking lever 26 is biasable relative to the calf guard 243 between a first position, namely, the locking position where the cam 261 is forced into the opening 245 and stopped against the track 241 to lock the calf guard 243 to the track 241, and a second position, namely, the unlocking position where the cam 261 is disengaged from the track 241 to unlock the calf guard 243 for allowing the calf guard 243 to be moved vertically along the track 241 to the desired elevation.

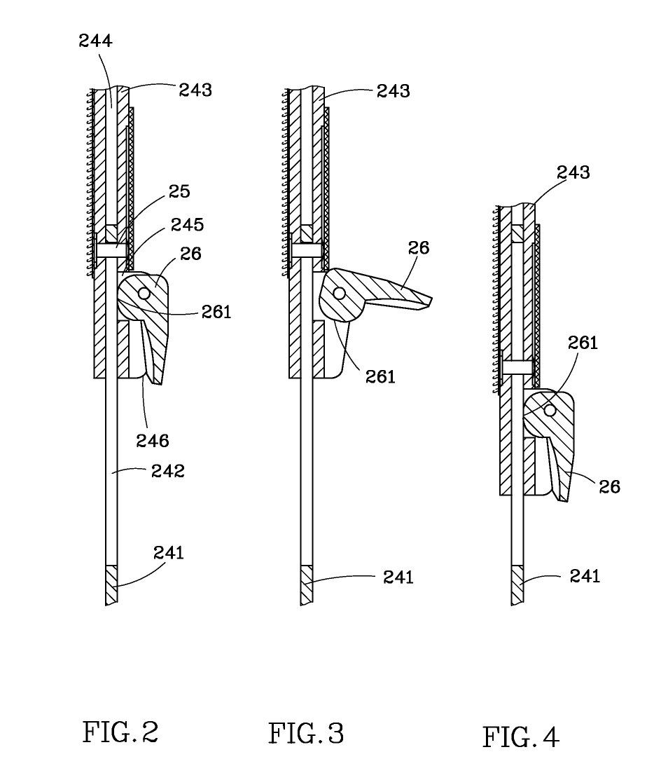

Referring to FIG. 2, when the locking lever 26 of the orthopedic shoe 2 is in the first (locking) position, the cam 261 is stopped against the track 241, locking the calf guard 243 to the track 241.

Referring to FIG. 3, when wanting to adjust the elevation of the calf guard 243, the user can bias the locking lever 26 from the first (locking) position to the second (unlocking) position to disengage the cam 261 from the track 241, for allowing the calf guard 243 to be moved vertically along the track 241 to the desired elevation.

Referring to FIG. 4, after the calf guard 243 has been adjusted to the desired elevation, the user can bias the locking lever 26 from the second (unlocking) position to the first (locking) position to force the cam 261 into engagement with the track 241, thereby locking the calf guard 243 to the track 241.

As stated above, the orthopedic shoe 2 allows the user to adjust the position of the calf guards 243 of the support devices 24 for comfort fit by means of operating the respective locking levers 26. Unlike conventional fixed size designs, the invention allows adjustment of the position of the calf guards 243 of the support devices 24 to fit the user's requirements.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An orthopedic shoe, comprising:
a shoe body;
at least one strap inserted through said shoe body for fastening said shoe body to a user's foot; and
at least one support device mounted on said shoe body to support the user's leg, said support device comprising a track affixed to said shoe body, a calf guard moveably coupled to said track and having a receiving chamber for receiving said track, and a locking lever pivoted to said calf guard and biasable relative to said calf guard between a locking position and an unlocking position, said locking lever having a cam forced into engagement with said track to lock said calf guard to said track when said locking lever is biased from said unlocking position to said locking position, said cam being disengaged from said track for allowing movement of said calf guard relative to said track when said locking lever is biased from said locking position to said unlocking position.

2. The orthopedic shoe as claimed in claim 1, wherein said calf guard comprises an opening disposed in communication with said receiving chamber; said cam of said locking lever is forced through said opening of said calf guard into engagement with said track when said locking lever is biased from said unlocking position to said locking position.

3. The orthopedic shoe as claimed in claim 2, wherein said calf guard comprises a plurality of protruding portions disposed around said opening; said locking lever is pivotally connected to said protruding portions of said calf guard.

4. The orthopedic shoe as claimed in claim 1, wherein said track comprises a sliding slot; said calf guard comprises a guide pin inserted through said sliding slot to guide sliding movement of said calf guard relative to said track along said sliding slot.
